(19) United States
(12) Patent Application Publication
(10) Pub. No.: US 2015/0157585 A1
(43) Pub. Date: Jun. 11, 2015

(54) PHARMACEUTICAL COMPOSITIONS

(71) Applicant: Corey Sipp, Macon, GA (US)

(72) Inventor: Corey Sipp, Macon, GA (US)

(21) Appl. No.: 14/170,059

(22) Filed: Jan. 31, 2014

Related U.S. Application Data
(60) Provisional application No. 61/914,807, filed on Dec. 11, 2013.

(51) Int. Cl. A61K 31/192; A61K 31/714; A61K 31/51; A61K 31/525; A61K 31/455; A61K 31/4415; A61K 31/519; A61K 31/4188; A61K 31/191; A61K 31/07; A61K 31/375; A61K 31/59; A61K 31/355; A61K 31/122; A61K 33/06; A61K 33/26; A61K 33/42; A61K 33/18; A61K 33/30; A61K 33/04; A61K 33/34; A61K 33/24; A61K 33/20; A61K 33/00; A61K 31/522

(52) U.S. Cl. CPC A61K 31/192; A61K 31/522; A61K 31/714; A61K 31/51; A61K 31/525; A61K 31/455; A61K 31/4415; A61K 31/519; A61K 31/4188; A61K 31/191; A61K 31/07; A61K 31/375; A61K 31/59; A61K 31/355; A61K 31/122; A61K 33/06; A61K 33/26; A61K 33/42; A61K 33/18; A61K 33/30; A61K 33/04; A61K 33/34; A61K 33/24; A61K 33/20; A61K 33/00

(57) ABSTRACT

Implementations of a pharmaceutical composition used to help treat pain, reduce inflammation and fatigue, provide vital nutrients, and increase alertness in humans are provided. In some implementations, the pharmaceutical composition can be comprised of an effective amount of a non-steroidal anti-inflammatory drug (NSAID), a multivitamin supplement, and an effective amount of caffeine sufficient to increase alertness, metabolic activity, and/or stave off drowsiness. The pharmaceutical composition can be formulated with any suitable nontoxic pharmaceutically acceptable inert carry material known to those skilled in the art and administered orally.

### TECHNICAL FIELD

This disclosure relates to implementations of a pharmaceutical composition.

### BACKGROUND

Non-steroidal anti-inflammatory drugs (NSAIDS) are used, in general, to treat both pain and inflammation. NSAIDS may be effective for the treatment of headaches, toothaches, backaches, arthritis, menstrual cramps, and stiff muscles and joints. Naproxen sodium, ibuprofen, and aspirin are all common NSAIDS which are available over-the-counter and/or through prescription. Many NSAIDs also have antipyretic and analgesic properties.

Caffeine is both a xanthine alkaloid and a stimulant. Acting on the central nervous system, caffeine helps ward off drowsiness and increases alertness. It is an additive found in many foods and medications.

Vital nutrients which humans must ingest to maintain proper cellular function are commonly referred to as Vitamins. Vitamins, in general, represent chemical compounds that cannot be synthesized in sufficient quantities by an organism. As such, the term “Vitamin” is conditional on both circumstance and on the cellular needs of the organism. Example vitamins for humans include vitamins from the A, B, C, D, E, and K complexes. Multivitamin supplements sold commercially frequently include dietary minerals and other nutritional elements, examples of which include calcium, phosphorus, potassium, sulfur, sodium, chlorine, magnesium, iron, cobalt, copper, zinc, molybdenum, iodine and selenium.

U.S. Patent Application Pub. No. 2010/0298258 (“'258 patent application”) discloses an analgesic composition used to treat pain and inflammation through a combination of analgesics, B vitamins, and caffeine. The '258 patent discloses that through the use of B vitamins and caffeine, the onset time of the analgesics is reduced and its pain relieving and/or anti-inflammatory effects enhanced.

However, the analgesic composition disclosed in the '258 application has several disadvantages. First, may not provide for the range of vitamins required for proper cellular function within humans. Instead, it is limited to B vitamins for their purported enhancement of the analgesics effectiveness. Second, the analgesic composition may not provide enough caffeine to stimulate the central nervous system sufficiently to increase alertness and stave off drowsiness.

### DETAILED DESCRIPTION

Implementations of a pharmaceutical composition used to help treat pain, reduce inflammation and fatigue, provide vital nutrients, and increase alertness in humans are provided. In some implementations, the pharmaceutical composition can be comprised of an effective amount of a non-steroidal anti-inflammatory drug (NSAID), a multivitamin supplement, and an effective amount of caffeine sufficient to increase alertness, metabolic activity, and/or stave off drowsiness. The pharmaceutical composition can be formulated with any suitable nontoxic pharmaceutically acceptable inert carry material known to those skilled in the art and administered orally.

In some implementations, the pharmaceutical composition comprises a therapeutically effective amount of the NSAID naproxen, or a pharmaceutically acceptable salt thereof (e.g., naproxen sodium), 150-220 milligrams (mg) of caffeine, and a multivitamin supplement. In some implementations, 200 milligrams (mg) of naproxen may be used. The pharmaceutical composition may be administered orally as a capsule, gelcap, tablet, pill, or related form.

Table 1 shows an example implementation of the pharmaceutical composition comprised of naproxen, caffeine and a multivitamin supplement according to the present disclosure. The pharmaceutical composition may be comprised of the following ingredients:

TABLE 1

| Components | Quantity | |
|---|---|---|
| Naproxen | 200 | mg |
| Caffeine | 150 | mg |
| Vitamin A | 3,500 | IU |
| Vitamin C | 60 | mg |
| Vitamin D | 400 | IU |
| Vitamin E | 30 | IU |
| Vitamin K | 25 | μg |
| (B₁) Thiamin | 1.5 | mg |
| (B₂) Riboflavin | 1.7 | mg |
| (B₃) Niacin | 20 | mg |
| Vitamin B₆ | 2 | mg |
| (B₉) Folic Acid | 400 | μg |
| Vitamin B₁₂ | 6 | μg |
| (B₇) Biotin | 30 | μg |
| (B₅) Pantothenic Acid | 10 | mg |
| Calcium | 200 | mg |
| Iron | 18 | mg |
| Phosphorus | 20 | mg |
| Iodine | 150 | μg |
| Magnesium | 50 | mg |
| Zinc | 11 | mg |
| Selenium | 55 | μg |
| Copper | 0.5 | mg |
| Manganese | 2.3 | mg |
| Chromium | 35 | μg |
| Molybdenum | 45 | μg |
| Chloride | 72 | mg |
| Potassium | 80 | mg |

In some implementations, an NSAID such as naproxen, ibuprofen, or aspirin (acetylsalicylic acid) is used to control pain and reduce swelling of the joints, muscles, or bones. In each instance, a therapeutically effective amount of the chosen NSAID is used, the quantity ranging between 100 to 1000 milligrams (mg), depending on the patient for whom the pharmaceutical composition is formulated. In some implementations, pharmaceutically acceptable salts of either ibuprofen or aspirin may be used.

In other implementations, a non-narcotic analgesic, such as acetaminophen, is used in place of an NSAID. An analgesic such as acetaminophen can be used to reduce pain.

The 150-220 milligram (mg) quantity of caffeine added to the pharmaceutical composition should be sufficient to increase alertness, metabolic activity, and stave off drowsiness in humans. In other implementations, the quantity of caffeine provided in the pharmaceutical composition is selected to enhance the effectiveness of the NSAID or to enhance the absorption of the vitamin(s). In some implementations, the quantity of caffeine in the pharmaceutical composition ranges between 100 to 400 milligrams (mg).

The multivitamin supplement included in the pharmaceutical composition may include vitamins A, B, C, D, E, and K. In some implementations, an amount of the following trace minerals may be added to the pharmaceutical composition: calcium, iron, phosphorous, iodine, magnesium, zinc, selenium, copper, manganese, chromium, molybdenum, chloride, potassium, or any combination of the foregoing.

In other implementations, B-complex vitamins including B₁ (Thiamin), B₂ (Riboflavin), B₃ (Niacin), B₅ (pantothenate), B_{6,} B₇ (Biotin), B₉ (Folic Acid) and B₁₂ are added to the pharmaceutical composition. In other implementations, vitamin B₁₂ is the only vitamin added to the pharmaceutical composition.

In some implementations, Table 1 for example, a combination of caffeine, NSAID(s), and vitamin(s) may produce an effect greater than the sum of their individual effects. For example, increased metabolic activity and stimulation of the central nervous system.

The various implementations of the pharmaceutical composition disclosed herein may be formulated with any suitable nontoxic pharmaceutically acceptable inert carrier material known to those skilled in the art. In some implementations, the pharmaceutical composition is formulated to be administered orally. In other implementations, the pharmaceutical composition is formulated to be administered rectally.

Reference throughout this specification to “an embodiment” or “implementation” or words of similar import means that a particular described feature, structure, or characteristic is included in at least one embodiment of the present invention. Thus, the phrase “in some implementations” or a phrase of similar import in various places throughout this specification does not necessarily refer to the same embodiment.

Many modifications and other embodiments of the inventions set forth herein will come to mind to one skilled in the art to which these inventions pertain having the benefit of the teachings presented in the foregoing descriptions and the associated drawings.

The described features, structures, or characteristics may be combined in any suitable manner in one or more embodiments. In the above description, numerous specific details are provided for a thorough understanding of embodiments of the invention. One skilled in the relevant art will recognize, however, that embodiments of the invention can be practiced without one or more of the specific details, or with other methods, components, materials, etc. In other instances, well-known structures, materials, or operations may not be shown or described in detail.

While operations are depicted in the drawings in a particular order, this should not be understood as requiring that such operations be performed in the particular order shown or in sequential order, or that all illustrated operations be performed, to achieve desirable results.

1. A pharmaceutical composition, comprised of:
a first composition comprising an effective amount of a non-steroidal anti-inflammatory drug;
a second composition comprising an effective amount of caffeine; and
a third composition comprised of a vitamin supplement.

2. The pharmaceutical composition of claim 1, wherein the first composition is further comprised of an analgesic.

3. The pharmaceutical composition of claim 2, wherein the analgesic of the first composition is non-narcotic.

4. The pharmaceutical composition of claim 1, wherein the first composition is comprised of between 100 mg to 1000 mg of at least one non-steroidal anti-inflammatory drug selected from a group consisting of ibuprofen, naproxen and aspirin.

5. The pharmaceutical composition of claim 1, wherein the second composition is comprised of between 100 mg to 400 mg of caffeine.

6. The pharmaceutical composition of claim 1, wherein the second composition is comprised of between 150 mg to 220 mg of caffeine.

7. The pharmaceutical composition of claim 1, wherein the third composition is comprised of vitamin B₁₂.

8. The pharmaceutical composition of claim 1, wherein the third composition is comprised of B-complex vitamins.

9. The pharmaceutical composition of claim 1, wherein the third composition is comprised of at least one vitamin selected from a group consisting of A vitamins, B vitamins, C vitamins, D vitamins, E vitamins, and K vitamins.

10. The pharmaceutical composition of claim 9, wherein the third composition is further comprised of at least one dietary element selected from a group consisting of calcium, phosphorus, potassium, sulfur, sodium, chlorine, magnesium, iron, cobalt, copper, zinc, molybdenum, iodine and selenium

11. The pharmaceutical composition of claim 1, wherein the third composition is comprised of A vitamins, B vitamins, C vitamins, D vitamins, E vitamins, and K vitamins.

12. A pharmaceutical composition, comprised of:
a first composition comprising 200 mg to 400 mg of the non-steroidal anti-inflammatory drug naproxen;
a second composition comprising an effective amount of caffeine; and
a third composition comprising A vitamins, B vitamins, C vitamins, D vitamins, E vitamins, and K vitamins.

13. The pharmaceutical composition of claim 12, wherein the third composition is further comprised of at least one dietary element selected from a group consisting of calcium, phosphorus, potassium, sulfur, sodium, chlorine, magnesium, iron, cobalt, copper, zinc, molybdenum, iodine and selenium.

14. The pharmaceutical composition of claim 12, wherein the second composition is comprised of 150 mg to 220 mg of caffeine.

15. The pharmaceutical composition of claim 14, wherein the first composition is comprised of 200 mg of naproxen.
